(19) United States
(12) Patent Application Publication
(10) Pub. No.: US 2010/0168081 A1
(43) Pub. Date: Jul. 1, 2010

(54) GALANTAMINE AS A NEUROPROTECTIVE DRUG FOR RETINAL GANGLION CELLS

(76) Inventor: Adriana Di Polo, Montreal (CA)

Correspondence Address: SMART & BIGGAR, 1000 DE LA GAUCHETIERE ST. W., SUITE 3300, MONTREAL, QC, H3B 4W5, CA

(21) Appl. No.: 12/063,554

(22) PCT Filed: Aug. 11, 2006
(86) PCT No.: PCT/CA2006/001334

§ 371 Date: Feb. 11, 2008

Related U.S. Application Data
(60) Provisional application No. 60/707,152, filed on Aug. 11, 2005.

(51) Int. Cl. A61K 31/55; A61P 25/00; A61P 27/02

(52) U.S. Cl. 514/215

(57) ABSTRACT

The present invention relates to the use of galantamine for neuroprotection of retinal ganglion cells.

This application claims priority to provisional application Ser. No. 60/707,152, filed Aug. 11, 2005, incorporated herein by reference.

### FIELD OF THE INVENTION

This invention relates to the administration or use of galantamine for the neuroprotection of retinal ganglion cells (RGCs).

### BACKGROUND OF THE INVENTION

The following review of the background of the invention is merely provided to aid in the understanding of the present invention and neither it nor any of the references cited within it are admitted to be prior art to the present invention. RGCs are neurons found in the retina inside the eye.

The retina is the inner layer of the eye at the back (posterior part) of the eyeball of vertebrates and some cephalopods. In cross section the mammalian retina is no more than 0.5 mm thick. It has five layers, three of nerve cells and two of synapses. The RGCs lie innermost in the retina while the photoreceptor cells (rods and cones) lie outermost. The retina contains photoreceptor cells that receive the light; the resulting neural signals then undergo complex processing by interneurons of the retina, and are transformed into action potentials in RGCs whose axons form the optic nerve. Therefore, RGCs are the sole neurons that convey visual information from the retina to the brain. The retina detects light and plays a significant part in visual perception. In embryonic development, the retina and the optic nerve originate as outgrowths of the brain. Indeed, the eye is considered to be an accessible part of the brain.

RGCs are a well-characterized neuronal population of the central nervous system (CNS), with cell bodies located in the inner retina and axonal processes along the optic nerve that reach specific targets in the brain. The cell bodies carry out all the main metabolic functions of the neuron, including gene expression and protein, metabolites and energy synthesis. The axons are responsible for the electrical transmission of visual information from the retina to the brain, as well as anterograde and retrograde transport of molecules essential for retinal ganglion cell (RGC) function.

The RGC is a key cell in the visual pathway; all visual information passing from the retina to the brain is encoded by these neural cells.

RGC death is the final common pathway for virtually all optic neuropathies. The initial insult in most optic nerve diseases is injury to the RGC axon, from either ischemia, inflammation, transection, or deformation. Optic nerve injury results in RGC apoptosis, partly by interfering with retrograde transport of target-derived neurotrophic factors.

Almost all optic neuropathies involve RGC axonal injury, except for a few disorders where the locus of injury is unknown. A method of preventing or treating RGC axonal injury would be applicable to a wide variety of diseases of the optic nerve, independent of the mechanism by which the nerve is injured. As many of these diseases have no effective therapy, determination of protective mechanisms could lead to innovative methods for their treatment. In particular, methods of preventing RGC death due to axonal injury could lead to innovative methods for their treatment.

There are different causes of RGC death. For example, age is a risk factor for RGC cell death. Ganglion cell diseases include the mitochondrially inherited Leber's hereditary optic neuropathy, temporary occlusion of the retinal artery, retrobulbar optic neuritis, dominant optic atrophy (DOA), and glaucomatous optic nerve disease (GOND), including glaucoma. In addition, there are a variety of diseases of the optic nerve and the retina that result in loss of ganglion cells, including optic neuritis and multiple sclerosis, optic neuropathies, orbital trauma, optic disk and nerve cancer, brain and spinal cord injury and age-related macular degeneration.

Leber's hereditary optic neuropathy (LHON) is an inherited disorder causing severe visual loss. Neuropathological studies of LHON have consistently shown degeneration of the retinal ganglion layer and optic nerve with ganglion cell axonal loss, occasionally with mild inflammation. There is no accepted treatment for this disease.

Dominant optic atrophy (DOA) is the most common form of autosomally inherited (non-glaucomatous) optic neuropathy. Patients with DOA present with an insidious onset of bilateral visual loss and they characteristically have temporal optic nerve pallor, centrocaecal visual field scotoma, and a color vision deficit, which is frequently blue-yellow. Evidence from histological and electrophysiological studies suggests that the pathology is confined to the RGC. There is no treatment available for this disease.

Visual loss from central retinal artery occlusion (CRAO) occurs from the loss of blood supply to the inner layer of the retina. There is a multitude of causes of CRAO, but patients typically present with sudden, severe, and painless loss of vision. Ganglion cells are damaged with CRAO. No particular treatment has proven unequivocally beneficial in treating CRAO. Examples of treatments used include, when acute, ocular-digital massage and/or anterior chamber paracentesis and/or administration of oral medications, such as acetazolamide, to lower intraocular pressure. Carbogen therapy, which is a carbon dioxide and oxygen combination, has been thought to help dilate vasculature and is used by some institutions. Hyperbaric oxygen therapy has also been described as a possible intervention.

Optic neuritis (ON) is defined as acute inflammation of the optic nerve, which can have many different causes, including infection (syphilis, mumps, measles), infiltrative/inflammatory disease (sarcoidosis, lupus), ischemic vascular disease (diabetes), and most commonly the demyelinating disease multiple sclerosis (MS). Current treatments include, for example, the administration of corticosteroids. However, there is no definitive evidence that treatment with steroids produces a more complete recovery than that which would have happened without treatment. An improved treatment is needed.

Ischemic Optic Neuropathy describes abnormalities of the optic nerve that occur as a result of ischemia, toxins, vascular and blood pressure abnormalities, and compression within the orbit. Ischemic optic neuropathy occurs when the optic nerve fails to receive a continuous, sufficient blood supply, and can lead to ganglion cell death. Toxic optic nerve damage, a type of ischemic optic neuropathy, can be caused by a large number of poisonous substances, drugs, nutritional deficiencies, metabolic disorders and radiation, which can lead to ganglion cell death. The triggering factor for an attack of acute ischemic neuropathy, even in the presence of arterio-sclerosis or other recognizable cardiovascular anomalies, is rarely identified. Management, therefore, presents complicated problems because ischemic optic neuropathy is not a diagnosis but a recognition of local anoxia of the anterior region of the optic nerve and the causes are both multiple and complex.

Age-related macular degeneration (AMD) is the leading cause of irreversible visual loss in the industrialized world, and is the leading cause of irreversible severe central visual loss in Caucasians 50 years and older in the U.S. The incidence and progression of the features of AMD increase significantly with age, with about 10% of patients 66-74 years of age having findings of AMD and increasing to 30% in patients 75 to 85 years of age.

Age-related macular degeneration (AMD) is a condition usually characterized by the deterioration of the macula and is also characterized by photoreceptor cell death. There are two forms of age-related macular degeneration: atrophic (dry) and neovascular (wet). Both forms of the disease can affect both eyes simultaneously. Vision can become severely impaired, affecting central vision rather than peripheral vision. The ability to see color is generally not affected, and total blindness from AMD is rare.

Macugen (pegaptanib sodium injection) and Lucentis are new drugs used for treating the wet form of age related macular degeneration. Macugen and Lucentis antagonize vascular endothelial growth factor, a protein that plays a role in the formation of new blood vessels, also known as angiogenesis. There is no known cure for the dry form of age related macular degeneration.

Preservation of ganglion cell layer neurons is thought to ameliorate age-related macular degeneration. As people age, the number of ganglion cells is reduced, therefore compounding the vision loss associated with age-related macular degeneration. It is possible that ganglion cells are affected in the very late stages of macular degeneration but this would be secondary as the primary cause of the disease affects only photoreceptors.

Glaucoma is the leading cause of blindness worldwide. Glaucoma is a degenerative eye disorder leading to loss of visual acuity principally due to the selective degeneration of RGCs. Elevated intraocular pressure (IOP) is a major risk factor in glaucoma although many patients continue to experience a progression of the disorder despite medications that lower IOP or glaucoma surgery.

Intraocular pressure is maintained by a balance between production and drainage of the aqueous humour, the fluid that fills the eye.

Several types of glaucoma have been described. In primary open-angle glaucoma, which accounts for about 60-70% of cases in the USA, the eye's filtration area also known as the trabecular meshwork does not function normally. If the drainage system does not function properly, pressure inside the eye builds up leading to optic nerve damage. Pigmentary glaucoma is an inherited type of open angle glaucoma. Angle-closure glaucoma, which accounts for about 10% of cases, results from an abnormality in eye structure. In most cases, the iris occludes the trabecular meshwork, preventing drainage of aqueous humor and raising intraocular pressure.

Glaucoma most frequently occurs after age 40, but can occur at any age. Persons of African heritage are more likely to develop open-angle glaucoma, and at an earlier age than Caucasians. Asians are more likely to develop closed-angle glaucoma.

Normal-tension glaucoma (NTG), also known as low-tension, or normal-pressure (between 12 and 22 mmHg), glaucoma, is not a rare condition. While it accounts for about 25% to 30% of US glaucoma cases, its diagnosis is more difficult than other forms of glaucoma that involve increase in intraocular pressure. Normal-tension glaucoma is prevalent in Japan, where twice as many people have normal-tension glaucoma as high-tension glaucoma.

Therefore, it is clear that optic nerve damage can occur in the absence of increased IOP. Having normal-tension glaucoma with optic nerve damage also carries a high risk for progression, even if eye pressure is reduced. Risk factors for normal tension glaucoma include Japanese ancestry and a family history of the disease. It is more common in women than in men. A family history of cardiovascular disease also increases the risk.

Traditional treatments for glaucoma have focused on decreasing intraocular pressure by a variety of means, including drugs, such as Latanoprost and Timolol and glaucoma surgery (e.g. trabeculoplasty).

AchE inhibitors, such as echothiophate iodide and physostigmine, have been used topically on the cornea to treat glaucoma by decreasing intraocular pressure. However, the toxicity of these drugs has resulted in their limited clinical use.

Importantly, many patients treated with intraocular pressure reducing drugs continue to experience vision loss. A significant group of glaucoma patients continue to have vision field loss in spite of responding well to drugs that reduce ocular pressure. Moreover, approximately 30% of all glaucoma patients never experience an increase in ocular pressure but still experience vision loss.

Following glaucoma surgery, the intraocular pressure is reduced temporarily, but returns to pre-surgical elevated levels within the next months to five years.

There are no neuroprotective drugs currently available for the treatment of diseases that cause RGC death or degeneration. The need for effective neuroprotective strategies applicable to such diseases has not been fulfilled by existing or prior art.

As many of these diseases involving RGC death have no effective therapy, determination of methods of RGC neuroprotection could lead to innovative methods for their treatment. In particular, methods of preventing RGC death following axonal injury could lead to innovative methods for their treatment.

### SUMMARY OF THE INVENTION

This invention responds to the medical need for neuroprotective therapies to halt or prevent the death of RGCs after trauma or in disease. The present invention demonstrates that galantamine can be used to promote neuroprotection of RGCs.

### BRIEF DESCRIPTION OF THE DRAWINGS

FIG. 1. Molecular structure of galantamine.

FIG. 2. Ocular hypertension surgery: Experimental protocol used to test the effect of galantamine on the survival of retinal ganglion cells (RGCs) in glaucoma after ocular hypertension surgery.

FIG. 3. Axotomy: Experimental protocol used to test the effect of galantamine on the survival of RGCs after axotomy of the optic nerve.

FIGS. 4A to 4C. Fluorescence photomicrographs of flat-mounted retinas showing DiI-labeled RGCs in intact and glaucomatous retinas treated with galantamine or saline at 5 weeks after ocular hypertension surgery. Images were taken from the superior central retina. Scale bar: 100 μm.

FIG. 4A is an intact retina.

FIG. 4B is a glaucomatous retina treated with galantamine.

FIG. 4C is a glaucomatous retina treated with Saline.

FIG. 5. Effect of galantamine on RGC survival in a preclinical model of glaucoma.

FIGS. 6A to 6C. Cross-sections of optic nerve segments from intact and glaucomatous eyes treated with or without galantamine at 5 weeks after ocular hypertension surgery.

FIG. 6A is a cross-section of optic nerve segment of an intact eye.

FIG. 6B is a cross-section of optic nerve segment of a glaucomatous eye.

FIG. 6C is a cross-section of optic nerve segment of a glaucomatous eye treated with galantamine.

FIG. 6D shows the numbers of RGC axons per optic nerve.

FIG. 7. Intraocular pressure (IOP) measurements made every other day for the entire duration of the experiment up to 35 days after ocular hypertension (OHT) surgery under daily treatment with intraperitoneal injection of galantamine.

FIGS. 8A to 8D. Representative images of flat-mounted retinas showing FluoroGold-labeled retinal ganglion cells (RGCs) in intact retinas (FIG. 8A), following intravitreal administration of galantamine (FIG. 8B), or saline (FIG. 8C) at two weeks after axotomy. Microglia that may have incorporated FluoroGold after phagocytosis of dying RGCs were distinguished by their morphology and excluded from Applicant's quantitative analyses. Scale bar: 100 μm. (FIG. 8D) Quantitative analysis of RGC survival following intravitreal injection of galantamine (solid bars) or saline (open bars) at 1 or 2 weeks after axotomy (n=4-5 rats per group). The density of RGCs in intact, untreated retinas (grey bar) is shown as reference.

FIG. 9. Alpha-bungarotoxin (αBgt), a selective blocker of alpha-7-nicotinic acetylcholine receptors, only partially blocked the neuroprotective effect of galantamine (GAL).

FIG. 10. The neuroprotective effect of galantamine on retinal ganglion cells (RGCs) was evaluated at 3 different doses at 5 weeks after ocular hypertension surgery: 0.5 mg/kg b.w., 3.5 mg/kg b.w., and 10 mg/kg b.w.

FIG. 11. Comparison of the neuroprotective effect of galantamine and donepezil on retinal ganglion cells (RGCs) at 5 weeks after ocular hypertension surgery.

FIG. 12. Comparison of the neuroprotective effect of galantamine and donepezil on retinal ganglion cells (RGCs) after axotomy of the optic nerve.

### DETAILED DESCRIPTION OF THE INVENTION

Galantamine hydrobromide (Reminyl) is an approved drug in many jurisdictions and is currently prescribed to Alzheimer's patients with mild to moderate cognitive deficits. In this invention, Galantamine has been demonstrated for the first time to be useful as a neuroprotective strategy for diseases causing RGC death.

Galantamine belongs to a class of drugs called acetylcholinesterase inhibitors (AchE inhibitors). This class of drugs includes tacrine, donepezil, rivastigmine and galantamine, all of which are currently being used to treat Alzheimer's patients. Importantly, Alzheimer's disease is characterized by a deficit in cholinergic neurotransmission that affects cholinergic neurons in the basal forebrain. It is principally the acetylcholinesterase inhibitory action of AchE inhibitors that is thought to treat Alzheimer's disease.

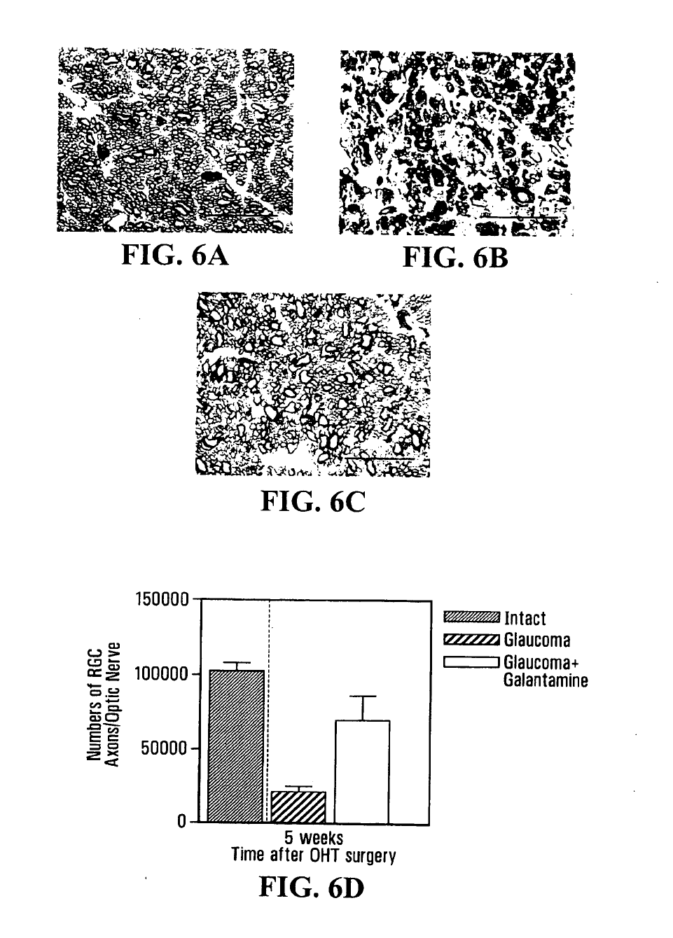

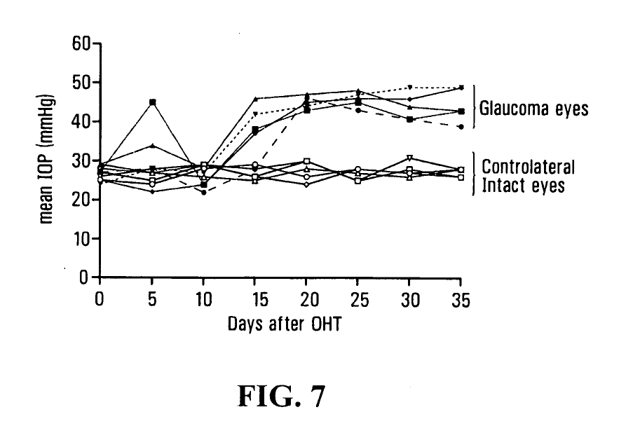

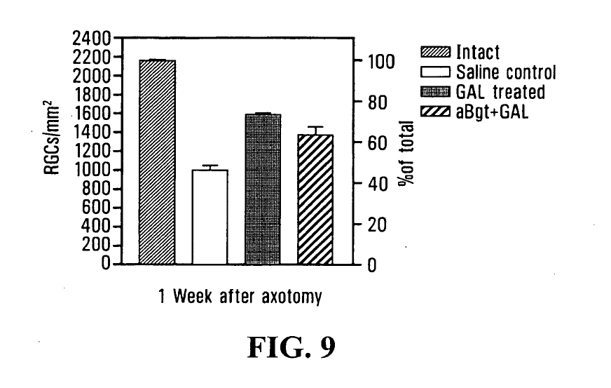

The categorization of galantamine as an AchE inhibitor distracts from its use as a neuroprotectant. Indeed, galantamine, applied topically on the cornea, has been demonstrated to reduce intraocular pressure. However, it has now been shown for the first time by the Applicant that systemic galantamine can achieve the proper concentration in the retina, leading to a robust neuroprotective effect on RGCs. This neuroprotective effect is observed without reducing intraocular pressure (see for example FIGS. 6 and 7, and Table I). This neuroprotectant effect of galantamine has been shown in the Examples to be separate and apart from its acetylcholinesterase inhibiting properties with respect to the alpha 7-nicotinic acetylcholine receptor (see FIG. 9). Furthermore, the Examples show that in particular, galantamine neuroprotects the RGC bodies and axons (see FIG. 6).

RGCs have a source of blood provided by capillaries that lie in the ganglion cell layer, which are supplied by the central retinal artery. Accordingly, drugs that achieve a sufficient blood concentration have the potential to be used to treat the RGCs.

Galantamine crosses the blood-brain barrier, and hence can access the blood supply of the RGCs. Accordingly, systemic administration of galantamine may be used to neuroprotect RGCs.

Systemic administration means any type of administration that accesses the general blood supply of the body, for example, oral administration, intravenous injections, intraperitoneal injections, intramuscular injections, intranasal administration and transepidermal patch administration are considered types of systemic administration. Systemic administration can include both enteral (oral) and parenteral (non-oral) administration.

Periocular and intraocular injections are useful modes of administering galantamine as they can achieve greater galantamine concentrations at the posterior part of the eye where galantamine neuroprotects RGCs, compared with topical ocular administration.

It has also been shown in the Examples, particularly in Example 2, that intravitreal injection of galantamine also neuroprotects RGCs. Intraocular and periocular administration of galantamine also neuroprotect RGCs.

Topical ocular administration includes eye drops and eye ointment.

Periocular means situated or occurring around the eye, as opposed to the term intraocular, which refers to within the eye. Periocular modes of administration include subconjunctival, subtenon, and retrobulbar administration.

Intraocular refers to within the eye. Intraocular administration includes intravitreal administration.

As mentioned previously, RGC (RGC) death is the final common pathway for virtually all optic neuropathies, and almost all optic neuropathies involve RGC axonal injury, except for a few disorders where the locus of injury is unknown. A method of preventing RGC axonal injury, such as systemic galantamine, intraocular galantamine or periocular galantamine, is applicable to a wide variety of diseases of the optic nerve, independent of the mechanism by which the nerve is injured. As many of these diseases have no effective therapy, systemic galantamine, intraocular galantamine and periocular galantamine are innovative methods for their treatment.

Such optic neuropathies, ganglion cell diseases and traumas suitable for treatment with galantamine include, but are not limited to, ganglion cell loss due to aging Leber's hereditary optic neuropathy (LHON), temporary occlusion of the retinal artery, retrobulbar optic neuritis, dominant optic atrophy (DOA), glaucomatous optic nerve disease (GOND), glaucoma, optic neuritis and multiple sclerosis, orbital trauma, optic disk and nerve cancer, brain and spinal cord injury and age-related macular degeneration.

Leber's hereditary optic neuropathy (LHON) is a mitochondrially inherited disease with male predominance.

LHON is characterized by bilateral optic atrophy with loss of central vision due to degeneration of the RGCs and their axons. There have been only a few neuropathological studies of LHON. These consistently show degeneration of the retinal ganglion layer and optic nerve with axonal loss, occasionally with mild inflammation. Others describe evidence of myelin splitting and reactive astrocytosis with vacuolar degeneration in the optic nerves. Additional findings include demyelination in the gracile columns of the spinal cord as well as some demyelination in peripheral nerves of the lower extremities.

Clinically, in LHON there is an acute onset of visual loss, first in one eye, and then a few weeks later in the other. This eventually evolves to very severe optic atrophy and permanent decrease of visual acuity. In the acute stage lasting a few weeks, the affected eye demonstrates an edematous appearance of the nerve fiber layer especially in the arcuate bundles and enlarged or telangectatic and tortuous peripapillary vessels (microangiopathy).

There are no neuroprotectant drugs currently approved for treatment of LHON. Galantamine is useful in treating this disease by neuroprotecting the RGCs.

Dominant optic atrophy (DOA) is the most common form of autosomally inherited (non-glaucomatous) optic neuropathy. Evidence from histological and electrophysiological studies suggests that the pathology is confined to the RGC. Therefore, galantamine is useful in neuroprotecting RGCs to treat this disease.

There are various types of trauma to RGCs where galantamine an help to neuroprotect the RGCs. For example, in central retinal artery occlusion (CRAO) vision loss occurs due to loss of blood supply to the inner layer of the retina. There is a multitude of causes of CRAO, but patients typically present with sudden, severe, and painless loss of vision. Ganglion cells are damaged with CRAO. No prior particular treatment has proven unequivocally beneficial in treating CRAO.

However, galantamine provides neuroprotection to RGCs and prevents vision loss due to trauma, particularly as evidenced in Example 2, which involved full transection of RGC axons (axotomy).

Optic neuritis (ON), also known as Demyelinating Optic Neuropathy and/or Retrobulbar Optic Neuritis, is defined as acute inflammation of the optic nerve. The condition is also known as retrobulbar neuritis because the nerve is located behind (“retro”) the globe of the eye. Retrobulbar neuritis is a form of optic neuritis in which the optic nerve, which is at the back of the eye, becomes inflamed. The inflamed area is between the back of the eye and the brain. The optic nerve contains RGC axon fibers that carry visual information from the retina to the brain. When these fibers become inflamed, visual signaling to the brain becomes disrupted, and vision is impaired. Vision loss can be minimal or the disease can result in complete blindness.

While etiologies of ON include infection (syphilis, mumps, measles), infiltrative/inflammatory disease (sarcoidosis, lupus), ischemic vascular disease (diabetes), the most common etiology is the demyelinating disease multiple sclerosis (MS). ON is the initial presenting sign in 20 to 25 percent of MS patients. Anywhere from 35 percent to 75 percent of patients who present with ON develop clinical MS. The risk of developing MS increases steadily during the first 10 years after the initial presentation of ON. The usual age range for the diagnosis of MS is 15 to 45 years. While some sources cite a predilection for females, the topic of sexual distribution remains controversial.

The clinical presentation of demyelinating optic neuropathy varies. Patients frequently present to the office with an acute loss of vision. The natural history of MS-related vision loss is rapidly progressive acuity loss for a period of 10 days, which then stabilizes and improves.

Multiple sclerosis is an acquired, multifactorial, inflammatory demyelinating disease, which affects the white matter located in the central nervous system. Myelin is responsible for speeding electrical impulses along nervous tissues. Loss of myelin greatly slows nervous conduction and leads to the neurologic deficits seen in MS.

Although the exact cause of MS is presently unknown, many theories regarding its etiology exist. The most common theories involve the immune system. The immune system attacks and destroys antigens by two different mechanisms. One mechanism, known as cellular immunity, involves a response by macrophages and T-cells. The other mechanism, governed by B-cells and antibodies is known as humoral immunity. Evidence suggests that the cellular immune response contributes to the loss of myelin. This patchy demyelination is thought to be caused by a deposition of mononuclear cells such as macrophages and B-cells in perivascular regions. Demyelinating optic neuropathy can damage the RGC axon fibers in both the visual and pupillary pathways. This damage interrupts nerve impulses within the pathways, producing decreased vision as well as an afferent pupillary defect.

Treating ON with corticosteroid therapy may not improve visual outcome after one year but may be found to increase the rate at which patients recover.

Treating ON with galantamine provides neuroprotection to RGCs to ameliorate the inflammation caused by ON, which affects these neural cells.

A number of other types of demyelinating disorders have been associated with ON. They are: acute transverse myelitis, Guillain-Barré syndrome, Devic's neuromyelitis optica, Charcot-Marie-Tooth syndrome, multifocal demyelinating neuropathy, and acute disseminated encephalomyelitis.

Other diseases that may cause optic neuropathy include syphilis, toxoplasmosis, histoplasmosis, tuberculosis, hepatitis, rubella, human immunodeficiency virus (HIV), Lyme borreliosis, familial Mediterranean fever, Epstein-Barr virus, herpes zoster ophthalmicus, paranasal sinus disorder, sarcoidosis, systemic lupus erythematosus, Bechet's disease, and diabetes.

These other diseases also benefit from treatment with galantamine, as galantamine provides neuroprotection to RGCs that are affected by such optic neuropathies.

As the visual dysfunction in MS is due to autoimmune destruction of myelin and not direct inflammation of the optic nerve tissue, this disease entity is best termed demyelinating optic neuropathy.

The average age of people who develop optic neuritis is 32. Most are female, and the vast majority also have pain when they move their eyes. Retrobulbar neuritis often is an early sign that someone has multiple sclerosis. Between 20% and 40% of the 25,000 people who develop optic neuritis in the United States each year will develop multiple sclerosis within 10 years.

Ischemic Optic Neuropathy is a type of Optic neuropathy. Optic neuropathy describes abnormalities of the optic nerve that occur as a result of ischemia, toxins, vascular and blood pressure abnormalities, and compression within the orbit. Ischemic disorders are termed “arteritic” when they occur secondary to inflammations of blood vessels, chiefly giant cell arteritis (temporal arteritis). They are termed nonarteritic when they are secondary to occlusive disease or other noninflammatory disorders of blood vessels. Optic neuropathy is divided into anterior, which causes a pale edema of the optic disk, and posterior, in which the optic disk is not swollen and the abnormality occurs between the globe and the optic chiasm.

Anterior ischemic optic neuropathy involves interruption of the blood flow in the short posterior ciliary arteries that supply the optic disk. This results in a severe loss of vision, altitudinal visual field defects, and a pale, swollen optic disk, with peripapillary hemorrhages. Ischemic anterior optic neuropathy usually causes a loss of vision that may be sudden or occur over several days. Patients are generally older than those with optic neuritis. There is often loss of the inferior visual field.

Posterior ischemic optic neuropathy is an uncommon type of neuropathy and diagnosis depends largely upon exclusion of other causes, chiefly stroke and brain tumor. There are altitudinal visual field defects sometimes combined with decreased visual acuity. Decreased blood flow in the minute pial vessels supplying the nerve, connective tissue disorders, diabetes mellitus, trauma, and radiotherapy to the orbit have all been described as causes. Impairment of visual acuity in ischemic optic neuropathy may vary from slight—with a corresponding decrease in color vision—to no light perception.

Age is the primary risk factor for anterior ischemic optic neuropathy. For posterior ischemic optic neuropathy, patients commonly have diabetes, hypertension, and hyperlipidemia, but any thrombotic condition capable of producing intracranial stroke can affect the ciliary arteries as well.

The main symptom of ischemic optic neuropathy is sudden loss of vision or reduced visual acuity. The cause of an attack of acute ischemic neuropathy even in the presence of arterio-sclerosis of other recognizable cardiovascular anomaly is rarely identified. Management, therefore, presents complicated problems because ischemic optic neuropathy is not a diagnosis but a recognition of local anoxia of the anterior region of the optic nerve and the causes are both multiple and complex.

Galantamine can be used in the treatment of ischemic optic neuropathy to protect the RGCs against damage due to lack of oxygen and blood supply due to the ischemia.

Galantamine can also be used to treat traumas involving brain and spinal cord injuries as a neuroprotectant. The retina of the eye is an outpost of the brain. Like the spinal cord, it is part of the Central Nervous System (CNS). The CNS includes the brain and spinal cord. Retinal neurons called ganglion cells carry signals from the retina to the brain. Their axons, together with supporting cells, form the optic nerve. Cutting or crushing the optic nerve, and thus the axons of the RGCs, has become an important model for injury and regeneration in the CNS. Therefore, if a drug, such as galantamine, neuroprotects the axons of the RGCs, this drug can also neuroprotect the brain and spinal cord.

Another target population that will benefit from this invention are patients with glaucoma. All glaucoma patients at risk of losing vision or that already experience visual defects, whether they have high or normal intraocular pressure, will benefit from treatment with galantamine. The results show that galantamine protects RGCs from ocular hypertensive damage. This drug protects RGC structure and preserves vision in glaucoma patients.

All types of glaucoma are characterized by RGC death. Since galantamine is a neuroprotectant that prevents RGC death, galantamine is effective in treating all types of glaucoma.

At present, there are no neuroprotective drugs available in the market for the treatment of glaucoma. Galantamine, a neuroprotective drug, is particularly useful for those glaucoma patients that have normal ocular pressure or that do not respond well to drugs that reduce ocular pressure.

Since galantamine crosses the retinal-blood barrier, it can therefore be taken orally and with few side effects. Galantamine can also be administered in other ways, such as by transepidermal patch, or other methods of systemic administration, or by intraocular or periocular administration, as described above.

From a clinical perspective, galantamine treatment confers neuroprotection on RGCs and preservation of vision in patients affected by glaucoma.

Galantamine can optionally be used in combination with drugs. In one embodiment, galantamine can be used in combination with drugs that control intraocular pressure, for example, beta-blocking agents, such as timolol, levobunolol, carteolol, and betaxolol; miotics, such as pilocarpine; carbonic anhydrase inhibitors, such as dorzolamide and brinzolamide; sympathomimetics, such as depivefrin, brimonidine, apraclonidine; and prostaglandin and like analogs, such as latanoprost, unoprostone, bimatoprost and travoprost.

The results show that galantamine may be used as a therapeutic agent for the treatment of RGC diseases.

Examples of pharmaceutically acceptable salts of galantamine are the sulfate, pyrosulfate, bisulfate, sulfite, bisulfite, phosphate, monohydrogenphosphate, dihydrogenphosphate, metaphosphate, pyrophosphate, hydrobromide, iodide, acetate, propionate, decanoate, caprate, caprylate, acrylate, ascorbate, formate, hydrochloride, dihydrochloride, isobutyrate, caproate, heptanoate, propiolate, propionate, phenylpropionate, salicylate, gluconate, stearate, glucarate, mesylate, tosylate, citrate, oxalate, malonate, succinate, suberate, sebacate, fumarate, malate, maleate, hydroxymaleate, mandelate, nicotinate, isonicotinate, cinnamate, hippurate, nitrate, phthalate, teraphthalate, butyne-1,4-dioate, butyne-1,4-dicarboxylate, hexyne-1,4-dicarboxylate, hexyne-1,6-dioate, benzoate, chlorobenzoate, methylbenzoate, hydroxybenzoate, methoxybenzoate, dinitrobenzoate, o-acetoxybenzoate, naphthalene-2-benzoate, phthalate, p-toluenesulfonate, p-bromobenzenesulfonate, p-chlorobenzenesulfonate, xylenesulfonate, phenylacetate, trifluoroacetate, phenylpropionate, phenylbutyrate, citrate, lactate, α-hydroxybutyrate, glycolate, tartrate, benzenesulfonate, methanesulfonate, ethanesulfonate, propanesulfonate, hydroxyethanesulfonate, 1-naphthalenesulfonate, 2-naphthalenesulfonate, 1,5-naphthalenedisulfonate, mandelate, tartrate, and the like.

Base addition salts include those derived from inorganic bases, such as ammonium or alkali or alkaline earth metal hydroxides, carbonates, bicarbonates, and the like.

Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, and the like.

Derivatives and stereoisomers of galantamine of the present invention include such compounds as described in U.S. Pat. No. 6,638,925, incorporated herein by reference in its entirety.

Analogs of galantamine can also be used as therapeutic agents for the treatment of RGC diseases. It has been shown that some analogs of galantamine could have equivalent or better pharmacologic profiles than galantamine, including duration of action, oral therapeutic index and pharmacokinetics. 6-O-Acetyl-6-O-demethylgalanthamine hydrochloride and 6-O-demethyl-6-O[(adamantan-1-yl)-carbonyl]galanthamine hydrochloride, are examples of analogs with therapeutic potential for the treatment of Alzheimer disease (Bores G M, et al. 1996).

Dosing is generally designed to achieve a concentration of galantamine in the brain in a particular range. The upper limit of that range can be 1800 nM, in another embodiment 1700 nM, in another embodiment 1600 nM, in another embodiment 1500 nM, in another embodiment 1400 nM, in another embodiment 1300 nM, in yet another embodiment 1200 nM, in another embodiment 1100 nM, in another embodiment 1000 nM, in another embodiment 900 nM, in another embodiment 800 nM, in another embodiment 700 nM, in another embodiment 600 nM, in another embodiment 500 nM, and in yet another embodiment, 400 nM. The lower limit of that range can be 100 nM, in another embodiment 200 nM, in another embodiment 300 nM, in another embodiment 400 nM, in another embodiment 500 nM, in another embodiment 600 nM, in another embodiment 700 nM, in another embodiment 800 nM, in another embodiment 900 nM, in another embodiment 1000 nM, in another embodiment 1100 nM, in another embodiment 1200 nM, in another embodiment 1300 nM, in another embodiment 1400 nM, in another embodiment 1500 nM and in yet another embodiment 1600 nM.

In another embodiment, galantamine is administered in a range of daily oral doses. The upper limit of the daily dose for an adult human patient (divided into two daily doses) can be about 32 mg, which is the total daily dose (given as twice daily dosing, each dose being 16 mg), in another embodiment about 30 mg (given as two 15 mg doses), in another embodiment about 28 mg (given as two 14 mg doses), in another embodiment about 26 mg (given as two 13 mg doses), in another embodiment about 24 mg (given as two 12 mg doses), in another embodiment about 22 mg (given as two 11 mg doses), in another embodiment about 20 mg (given as two 10 mg doses), in another embodiment about 18 mg (given as two 9 mg doses), and in another embodiment about 16 mg (given as two 8 mg doses). The lower limit of the daily dose (divided into two daily doses) can be about 8 mg (given as twice daily dosing, each dose being 4 mg), in another embodiment about 10 mg (given as two 5 mg doses), in another embodiment about 12 mg (given as two 6 mg doses), in another embodiment about 14 mg (given as two 7 mg doses), in another embodiment about 16 mg (given as two 8 mg doses), in another embodiment about 18 mg (given as two 9 mg doses), in another embodiment about 20 mg (given as two 10 mg doses), in another embodiment about 22 mg (given as two 11 mg doses), in another embodiment about 24 mg (given as two 12 mg doses), in another embodiment about 26 mg (given as two 13 mg doses), in another embodiment about 28 mg (given as two 14 mg doses), in another embodiment about 30 mg (given as two 15 mg doses), and in another embodiment about 32 mg (given as two 16 mg doses).

In one embodiment, galantamine is administered systemically. The Examples show that intraperitoneal (i.p.) administration of galantamine resulted in neuroprotection and did so without reducing intraocular pressure (see FIG. 7). Oral administration or other types of systemic administration of galantamine will produce the same result.

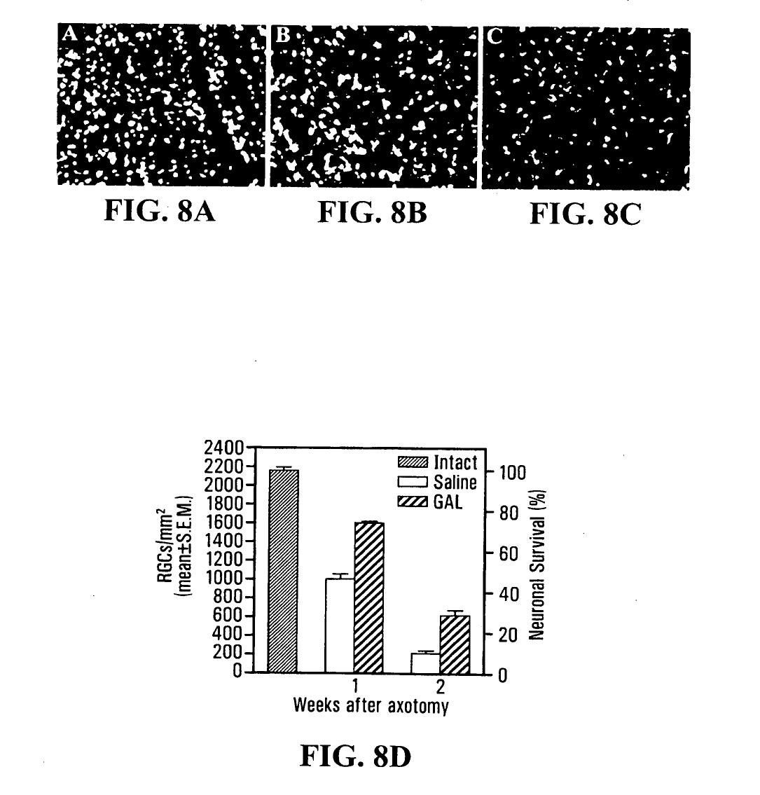

In another embodiment, galantamine is administered intraocularly or periocularly. The Examples show that intravitreal administration of galantamine resulted in neuroprotection (see FIG. 8D and FIG. 9).

In various embodiments, galantamine is used therapeutically in formulations or medicaments to neuroprotect RGCs. The invention provides corresponding methods of medical treatment, in which a therapeutic dose of galantamine is administered in a pharmacologically acceptable formulation, e.g. to a patient or subject in need thereof. Accordingly, the invention also provides therapeutic compositions comprising galantamine, and a pharmacologically acceptable excipient or carrier. In one embodiment, such compositions include galantamine in a therapeutically or prophylactically effective amount sufficient to neuroprotect RGCs. The therapeutic composition may be soluble in an aqueous solution at a physiologically acceptable pH.

A “therapeutically effective amount” refers to an amount effective, at dosages and for periods of time necessary, to achieve the desired therapeutic result, such as a reduction in RGC death. A therapeutically effective amount of galantamine may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing ganglion cell death or inhibiting the rate of RGC degeneration-related disease onset or progression. A prophylactically effective amount can be determined as described above for the therapeutically effective amount. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, transepidermal, periocular, intravitreal, intraocular, intranasal, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, galantamine can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound (galantamine) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of galantamine plus any additional desired ingredient from a previously sterile-filtered solution thereof. In accordance with an alternative aspect of the invention, galantamine may be formulated with one or more additional compounds that enhance the solubility of the galantamine.

In accordance with another aspect of the invention, therapeutic compositions of the present invention, comprising galantamine, may be provided in containers or commercial packages which further comprise an intraocular delivery device or system for delivery of said compositions to RGCs in RGC degeneration-related diseases.

In one embodiment, such intraocular delivery system comprises the pSivida drug delivery system. pSivida owns the rights to develop and commercialize a modified form (porosified or nano-structured) of silicon known as BioSilicon™, which has applications in drug delivery, wound healing, orthopedics, and tissue engineering. Retisert™ (fluocinolone acetonide intravitreal implant, owned by Bausch & Lomb), uses the pSivida drug delivery system. Retisert™ is a treatment for chronic, non-infectious uveitis affecting the posterior segment of the eye, a debilitating eye disease that is the third largest cause of blindness in the U.S., affecting 175,000 people.

In another embodiment, such intraocular delivery system comprises the I-vation™ (owned by SurModics Inc, Minneapolis, Minn.). The I-vation™ sustained drug delivery system is an implantable helical coil that utilizes SurModics' proprietary drug delivery polymer coatings for sustained release of therapeutics into the posterior chamber of the eye. The implant is capable of providing long-term drug delivery, thus replacing frequent intraocular injections, the current standard of care.

In yet another embodiment, such intraocular delivery system comprises Posurdex™ (owned by Allergan, Inc., Irvine, Calif.). Posurdex™ is a biodegradable, micro-sized intraocular drug delivery system that is designed to release therapeutic levels of an active drug inside the eye. Posurdex™ has been successfully used for intraocular delivery of dexamethasone, a marketed corticosteroid known for its safety and potent anti-inflammatory effect, for approximately one month. As the dexamethasone is released, the polymer matrix dissolves and is naturally absorbed by the body.

In accordance with another aspect of the invention, therapeutic compositions of the present invention, comprising galantamine, may be provided in containers or commercial packages which further comprise instructions for use of galantamine for the neuroprotection of RGCs in RGC degeneration-related diseases.

Accordingly, the invention further provides a commercial package comprising galantamine or the above-mentioned composition together with instructions for the neuroprotection of RGCs.

The invention further provides a use of galantamine for neuroprotection of RGCs. The invention further provides a use of galantamine for the preparation of a medicament for neuroprotection of RGCs.

In another aspect, the invention further provides that galantamine may be synthetic or may be derived from natural sources such as the Amaryllidaceae, including the narcissi, crinum or galanthus species. Particularly suitable are Narcissus pseudonarcissus “Carlton” or the Asian climber Crinum amabile or the snowdrop or Leucojum aestivum. According to one aspect of the present invention, galantamine is derived from a natural source.

All patents, patent applications and publications mentioned herein, both supra and infra, are hereby incorporated by reference.

While the invention has been described with reference to certain specific embodiments and will be described in the following Examples, it is understood that it is not to be so limited since alterations and changes may be made therein which are within the full and intended scope of the appended claims.

Now in order to more particularly define some embodiments of the present invention, the following Examples provide details of specific compounds of the invention, methods of producing the same and results from testing such compounds.

### Example I

This example demonstrates that galantamine can promote RGC survival in a pre-clinical rat model of glaucoma

### 1) Experimental Protocol

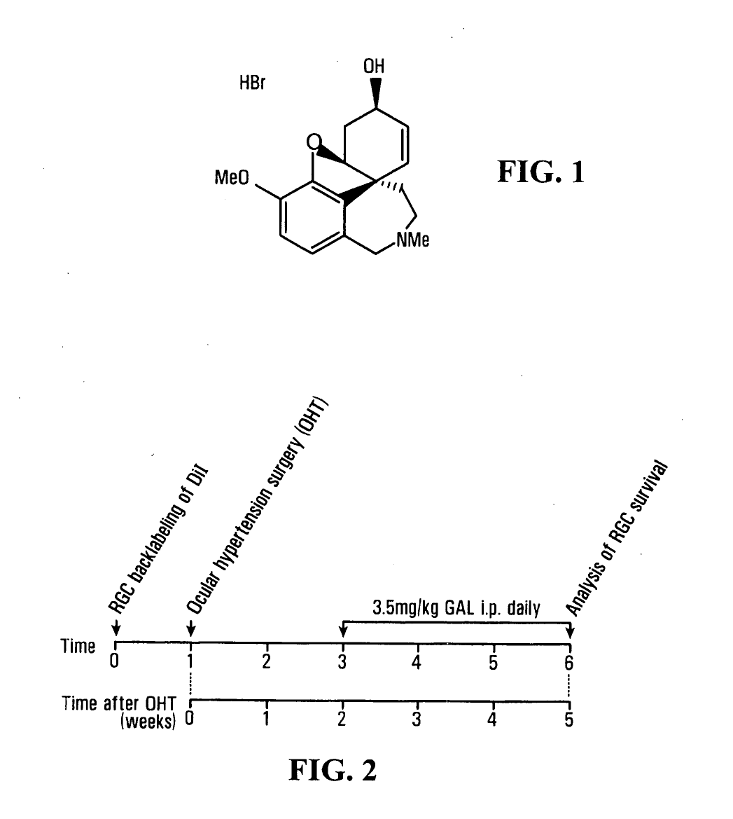

The experimental protocol used to test the effect of galantamine (the molecular structure of which is shown in FIG. 1) on the survival of RGCs in glaucoma is provided in FIG. 2. RGCs were first labeled by application of the retrograde fluorescent tracer (DiI) to the superior colliculus, followed by induction of ocular hypertension. Two weeks after ocular hypertension surgery, animals were subjected to daily treatment with galantamine −3.5 mg/kg body weight, intraperitoneal administration (i.p.). Analysis of RGC survival was carried out at 5 weeks after ocular hypertension surgery.

### 2) Dose of Galantamine and Route of Delivery In Vivo

An important issue is the concentration of galantamine used in vivo and its comparison with clinically attainable levels in the brain or retina of human patients. Galantamine is a small molecule capable of crossing the blood-brain barrier. Galantamine brain levels in Alzheimer's disease patients can be deduced from PET imaging studies using a tracer (e.g. PMP or MP4A) specific for the activity of brain AchE inhibition.

### 2a) Intraperitoneal Administration of Galantamine

In previous studies conducted in Alzheimer's patients, galantamine at a maintenance dose of 16 or 24 mg/day resulted in maintenance or improvement of basic and instrumental activities of daily living, detectable after 5 months of treatment, regardless of dementia severity. More specifically, Alzheimer's disease patients treated for 3 weeks or 3 months with 24 mg of galantamine had an estimated brain concentration of functionally available galantamine between 500-1200 nM (Bores et al. 1996; Thomsen and Kewitz 1990). A recent study indicated that daily doses between 1 and 5 mg/kg body weight (b.w.) galantamine in mice achieve brain levels in the 200-1000 nM range (Geerts et al. 2005). Based on this, a dose of 3.5 mg/kg b.w., administered intraperitoneally, was selected for this Example. Therefore, two weeks after ocular hypertension surgery, animals received daily intraperitoneal (i.p) injections of galantamine at a dose of 3.5 mg/kg b.w. as shown in FIG. 2.

### 3) Experimental Animals

All surgeries were performed in adult male Brown Norway rats, retired breeders, between 10-12 months of age (300-400 g), under general anesthesia by intraperitoneal injection of 1 ml/kg b.w. standard rat cocktail consisting of ketamine (100 mg/ml), xylazine (20 mg/ml) and acepromazine (10 mg/ml).

### 4) Retrograde Labeling of RGCs

For neuronal survival experiments, RGCs were retrogradely labeled with 3% DiI (1,1′-dioctadecyl-3,3,3′,3′-tetramethyl-indocarbocyanine perchlorate; Molecular Probes, Junction City, Oreg.), a fluorescent carbocyanine marker that persists for several months without fading or leakage and does not interfere with the function of labeled cells (Honig and Hume 1986; Vidal-Sanz et al. 1988). For retrograde labeling, both superior colliculi, the main targets of RGCs in the brain (Linden and Perry 1983), were exposed and a small piece of gelfoam (Pharmacia and Upjohn Inc., Mississauga, ON) soaked in Dii was applied to their surface. Seven days after DiI application, the time required for labeling the entire RGC population, animals were subjected to ocular hypertension surgery as described below.

### 5) Ocular Hypertension Surgery

Unilateral and chronic elevation of IOP was induced as previously described (Morrison et al. 1997) using a method that involves injection of a hypertonic saline solution into an episcleral vein. This model of ocular hypertension leads to inner retinal atrophy, optic nerve degeneration, and optic nerve head remodeling similar to that seen in human, age-related glaucoma (Morrison et al. 1997). More importantly, this model is considered as one of the best preclinical models of human glaucoma.

All the animals involved in this study received only a single saline vein injection. The eye selected for the procedure was adapted with a plastic ring in the ocular equator to confine the injection to the limbal plexus. A microneedle (30-50 μm in diameter) was used to inject 50 μl of sterile 1.85 M NaCl solution through one episcleral vein. The plastic ring temporarily blocked off other episcleral veins forcing the saline solution into the Schlemm's canal to create isolated scarring. Animals were kept in a room with constant low fluorescent light (40-100 lux) to stabilize circadian IOP variation (Moore et al. 1996).

### 6) Measurement of Intraocular Pressure (IOP)

IOP from glaucomatous and normal (contralateral) eyes was measured in awake animals using a calibrated tonometer (TonoPen XL, Medtronic Solan, Jacksonville, Fla.). Ten to fifteen consecutive readings per eye were taken and averaged to obtain an accurate daily IOP measurement. IOP was measured daily for two weeks after ocular hypertension surgery, and then every other day for the entire duration of the experiment. The mean IOP (mm Hg±S.E.M.) per eye was the average of all IOP readings since the onset of pressure elevation. The maximum IOP measured in each individual eye, glaucomatous or normal contralateral eye, was defined as the peak IOP and this value was used to estimate the mean peak IOP for each group. The delta positive integral IOP was calculated as the area under the IOP curve in the glaucomatous eye minus that of the fellow normal eye from ocular hypertension surgery to euthanasia. Integral IOP represents the total, cumulative IOP exposure throughout the entire experiment.

### Example 2

This example demonstrates that galantamine can promote RGC survival after traumatic optic nerve injury.

### 1) Experimental Protocol

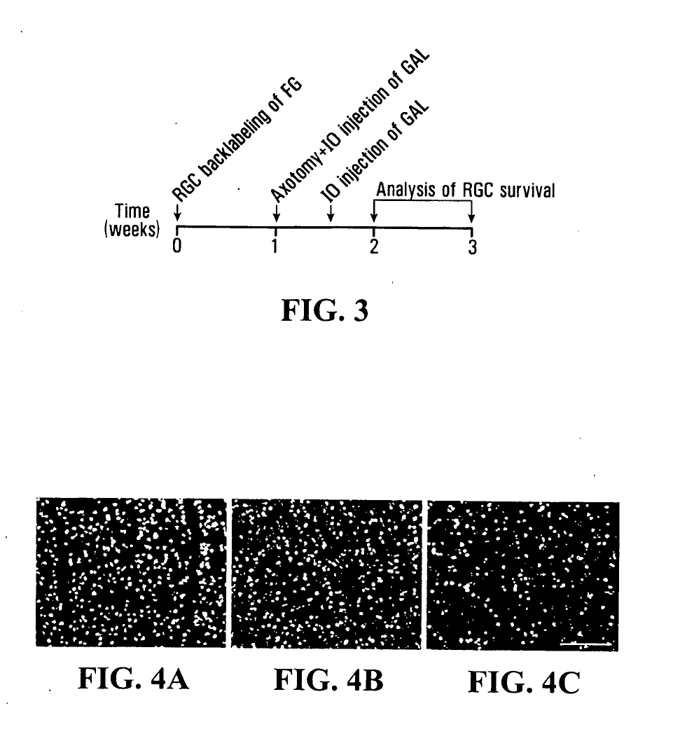

Experimental protocol used to test the effect of galantamine on the survival of RGCs after axotomy of the optic nerve is provided in FIG. 3. RGCs were first labeled by application of the retrograde fluorescent tracer FluoroGold to the superior colliculus, followed by transection of the optic nerve. Then, two intravitreal injections of galantamine (purchased from Janssen-Cilag) were performed: one immediately after optic nerve cut and, a second, 4 days later. Animals were euthanized 1 and 2 weeks after optic nerve injury and RGC survival was analyzed.

### 2) Intravitreal Injection of Galantamine

The effect of galantamine on the protection of RGCs after optic nerve cut was also assessed by multiple intravitreal injection of galantamine performed at the time of axotomy and 4 days later, as shown in FIG. 3. A range of galantamine concentrations between 1 and 200 μM was tested in this model to determine the optimal concentration of galantamine that confers RGC neuroprotection. About 5 μl (intravitreal) of galantamine was typically injected at each of the concentrations tested. The final concentration inside the eye can be calculated by assuming that the intravitreal volume in an adult rat is ˜60 μl.

### 3) Intraocular Injection of Alpha-Bungarotoxin

Alpha-bungarotoxin (10 μM), a selective blocker of alpha 7 nicotinic acetylcholine receptors, was injected into the vitreous chamber of one eye as described in section 2. Alpha-bungarotoxin was injected simultaneously with galantamine.

### 4) Experimental Animals

All surgeries were performed in female Sprague-Dawley rats (−200 g), under general anesthesia by intraperitoneal injection of 1 ml/kg b.w. standard rat cocktail consisting of ketamine (100 mg/ml), xylazine (20 mg/ml) and acepromazine (10 mg/ml).

### 5) Retrograde Labeling of RGCs

For experiments using the optic nerve axotomy model, RGCs were retrogradely labeled with 2% FluoroGold (Fluorochrome, Englewood, Colo.).

### 6) Optic Nerve Axotomy

Following application of the retrograde tracer, a group of animals were subjected to transection of the left optic nerve at 0.5-1 mm from the back of the eye avoiding injury to the ophthalmic artery. Rats were euthanized at 7 or 14 days post-lesion by intracardial perfusion with 4% paraformaldehyde. Both the left (optic nerve lesion) and right (intact control) retinas were dissected for quantification of surviving neurons as described below. The vasculature of the retina was routinely monitored by fundus examination and animals showing signs of compromised blood supply were eliminated from the analysis.

### Example 3

The following provides the method of quantification of surviving RGC bodies and quantification of surviving RGC axons for the experiments described in Example 1 and Example 2, above.

### 1) Quantification of Surviving RGC Bodies

Analysis of RGC survival was carried out at 5 weeks after ocular hypertension surgery. Quantification of RGC bodies or axons was always performed in duplicate and in a masked fashion. For RGC density counts, rats were deeply anaesthetised and perfused intracardially with 4% paraformaldehyde (PFA) in 0.1 M phosphate buffer and both eyes were immediately enucleated. Retinas were dissected and flat-mounted on a glass slide with the ganglion cell layer side up. Under fluorescence microscopy, DiI-labeled or FluoroGold-labeled neurons were counted in 12 standard retinal areas as previously described (Cheng et al. 2002; Sapieha et al. 2003).

### 2) Quantification of Surviving RGC Axons

For axonal counts, animals received an intracardiac injection of heparin (1,000 u/kg b.w.) containing sodium nitroprusside (10 mg/kg b.w.) followed by intracardiac perfusion with 2% PFA and 2.5% glutaraldehyde in 0.1 M phosphate buffer. Optic nerves were dissected, fixed in 2% osmium tetroxide, and embedded in epon resin. Semi-thin sections (0.7 μm thick) were cut on a microtome (Reichert, Vienna, Austria) and stained with 1% toluidine blue. RGC axons were counted in five non-overlapping areas of each optic nerve section, encompassing a total area of 5,500 μm² per nerve. The five optic nerve areas analyzed included one in the center of the nerve, two peripheral dorsal and two peripheral ventral regions. The total surface area per optic nerve cross section was measured using the Northern Eclipse image analysis software, and this value was used to estimate the total number of axons in each optic nerve. Applicant estimated that 2% of the total number of axons in the optic nerve and 1.8% of the total number of RGCs, with respect to the total number of axons and RGCs found in normal retinas, were sampled in Applicant's quantitative analysis.

### Example 4

This example demonstrates that galantamine can promote RGC survival in glaucoma experimental model.

### 1) Galantamine Protected RGC Soma in Glaucomatous Eyes

Applicant's results demonstrate that daily treatment with 3.5 mg/kg b.w. galantamine (purchased from Janssen-Cilag) administered intraperitoneally resulted in striking protection of RGCs from ocular hypertension damage. The neuroprotective effect of galantamine led to higher neuronal densities and better preservation of cellular integrity than in saline-treated control eyes (FIG. 4).

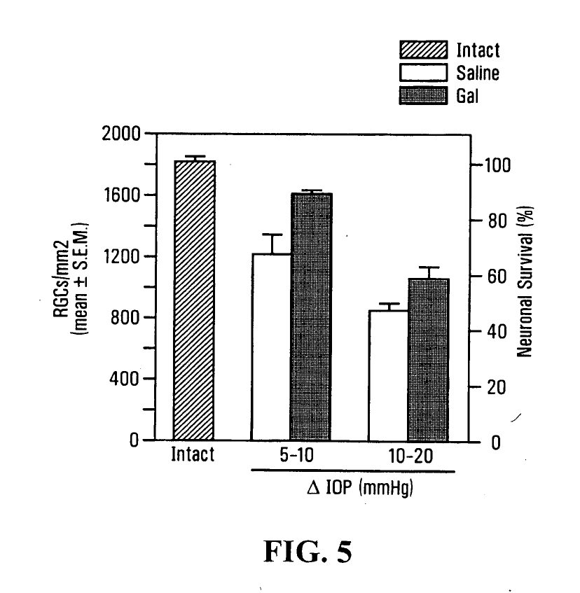

In animals with intraocular pressure increase between 5-10 mm Hg (FIG. 5), galantamine preserved 90% of RGCs at 5 weeks after ocular hypertension surgery (n=7) compared with 65% that survived in control eyes that received daily injections of saline (n=3). A marked neuroprotective effect of galantamine at 5 weeks after ocular hypertension surgery was also apparent in a group of animals with higher intraocular pressure (ΔIOP=10-20 mm Hg, n=14, FIG. 5) with respect to controls (n=4). These data clearly indicate that galantamine protects RGC soma in glaucoma.

### 2) Galantamine Reduced RGC Axon Damage in Glaucoma

Glaucoma is characterized by the degeneration of RGC axons in the optic nerve followed by the progressive loss of cell bodies (Quigley 1999; Schwartz et al. 1999). Hence, Applicant investigated the effect of galantamine on RGC axon protection following ocular hypertension damage. FIG. 6 provides cross-sections of optic nerve segments from intact and glaucomatous eyes treated with or without galantamine at 5 weeks after ocular hypertension surgery. Optic nerve cross-sections from galantamine-treated eyes displayed a larger number of axonal fibers with normal morphology (FIG. 6) compared to untreated, glaucomatous eyes, which showed extensive axon degeneration including disarray of fascicular organization and degradation of myelin sheaths. Quantification of RGC axons demonstrated that galantamine protected a significant number of axons in the optic nerve at 5 weeks after ocular hypertension surgery. Together, these results indicate that galantamine protects RGC axons in glaucoma.

### 3) The Neuroprotective Effect of Galantamine was not Caused by Reduction of Intraocular Pressure

Applicant tested whether intraperitoneal administration of galantamine reduced intraocular pressure, which could account for the neuroprotective effect. For this purpose, Applicant measured intraocular pressure every other day for entire duration of daily treatment with galantamine up to 35 days after ocular hypertension (OHT) surgery. Applicant's results demonstrate that intraperitoneal injection of galantamine at a dose of 3.5 mg/kg b.w. did not reduce intraocular pressure as shown in FIG. 7. These results clearly indicate that the neuroprotective effect of galantamine is not caused by reduction of intraocular pressure.

4) Galantamine Protected RGCs from Death After Traumatic Optic Nerve Injury

To further confirm that galantamine has a neuroprotective effect on injured RGCs that is independent of reduction of intraocular pressure, Applicant examined its survival effect in a different model of RGC death: axotomy of the optic nerve. Applicant's data demonstrate that two intraocular injections of galantamine (100 μM), provided at the time of axotomy and 4 days later, led to a dramatic increase in the density of RGCs after optic nerve injury (FIG. 8). For example, galantamine treatment led to the survival of >75% of axotomized RGCs at 1 week after optic nerve injury (n=5), ˜30% of RGCs remained alive at 2 weeks of nerve lesion (n=4), a time when ˜90% of these neurons are lost in a control group treated with saline (n=5) (ANOVA, p<0.001).

These data confirm that galantamine exerts a potent neuroprotective effect on RGCs that is independent of changes in intraocular pressure. In addition, these results show that galantamine can effectively protect neurons against traumatic nerve injury. Given that RGCs are a prototypical CNS neuronal population, this finding suggests that galantamine may be used for the prevention of neuronal death after traumatic injury in the adult brain or spinal cord.

### 5) Alpha-bungarotoxin, a Selective Blocker of Alpha-7-Nicotinic Acetylcholine Receptors, Only Partially Blocked the Neuroprotective Effect of Galantamine.

Galantamine is a modest Acetylcholinesterase inhibitor that also modulates the alpha-7 nicotinic acetylcholine receptor, as an allosteric potentiating ligand (Samochocki et al. 2003). To determine the contribution of the alpha-7 nicotinic acetylcholine receptor in the neuroprotective effect of galantamine, Applicant used alpha-bungarotoxin, a selective blocker of this receptor.

Alpha-bungarotoxin was injected simultaneously with galantamine and its effect was tested in the model of RGC axotomy (FIG. 9). Applicant's results show that Alpha-bungarotoxin blocked ˜10-15% of the total neuroprotective effect of galantamine. These results indicate that activation of the alpha-7 nicotinic acetylcholine receptor contributes only partially to the neuroprotective effect of galantamine. These data also suggest that other receptors or molecules, as yet unidentified, may participate in galantamine-induced RGC survival.

### 6) Neuroprotective Effect of Galantamine on RGCs at Different Doses.

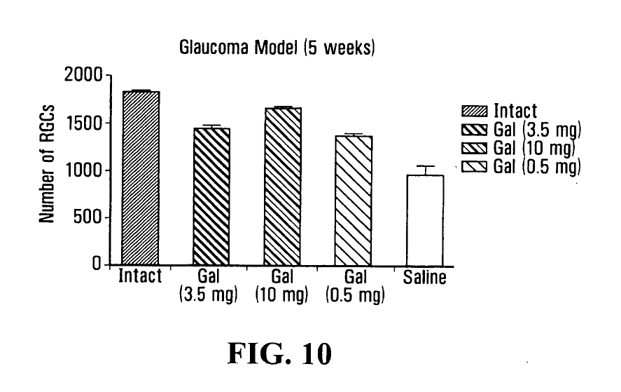

FIG. 10 shows the quantitative analysis of the number of retinal ganglion cells (RGCs, mean±S.E.M.) per mm² of retina after treatment with 3 different systemic doses of galantamine at 5 weeks after ocular hypertension (OHT) surgery.

Galantamine was administered daily by intraperitoneal injection of 0.5 mg/kg b.w. (n=2), 3.5 mg/kg b.w. (n=14), or mg/kg b.w. (n=2). All these doses were significantly more neuroprotective than saline treatment (used as control).

These data indicate that, when compared with saline-treated control eyes, galantamine was effective at promoting survival of retinal ganglion cell in glaucoma at all doses tested (and more particularly at doses between 0.5-10 mg/kg b.w., and between 0.5-3.5 mg/kg b.w., and between 3.5-10 mg/kg b.w.).

### Example 5

This example compares the neuroprotective effect of two AchE inhibitors, galantamine and donepezil, in promoting RGC survival in both glaucoma experimental model and in optic nerve injury (axotomy).

### 1) Effect of Systemic Administration of Galantamine or Donepezil on Intraocular Pressure (IOP) in Experimental Glaucoma.

The effect of systemic administration of galantamine (3.5 mg/kg b.w.) or donepezil (3.5 mg/kg b.w.), another acetylcholinesterase inhibitor, on intraocular pressure (IOP) in experimental glaucoma was examined at 5 weeks after OHT surgery.

Table I shows that baseline mean IOP in both eyes prior to ocular hypertension surgery was ˜27 mm Hg, which is a typical measurement in awake rats that are housed in a constant light environment to stabilize circadian IOP variations. Mean sustained pressure elevation among galantamine, saline and No Treatment groups was ˜17 mm Hg, well within the range of IOP increase observed in this model. The integral IOP, which represents the total IOP elevation experienced by the glaucomatous eye with respect to control eyes throughout the entire duration of the experiment was also consistent among the galantamine, saline and No treatment groups. In contrast, the group treated with donepezil showed a significant reduction in the mean IOP (9.7 mm Hg) and integral IOP (246 mm Hg).

TABLE I

| | | Mean IOP | | | Integral |
|---|---|---|---|---|---|
| | | (mmHg) | | | IOP |
| Group | N | Glaucoma | Control | Difference | (mmHg) |
| Galantamine | 24 | 40.7 ± 0.8 | 23.7 ± 0.2 | 17 ± 0.8 | 321 ± 10 |
| (3.5 mg/kg | | | | | |
| b.w.) | | | | | |
| Donepezil | 5 | 31.7 ± 1.5 | 22.07 ± 0.6 | 9.7 ± 0.8 | 246 ± 7 |
| (3.5 mg/kg | | | | | |
| b.w.) | | | | | |
| Saline | 12 | 39 ± 1.6 | 22.4 ± 0.3 | 16.7 ± 1.2 | 295 ± 18 |
| No treatment | 12 | 41.4 ± 1.5 | 23.8 ± 0.2 | 17.6 ± 1.7 | 311 ± 16 |

These data clearly demonstrate that daily treatment with galantamine did not change the mean IOP increase in glaucomatous or normal eyes. In contrast, donepezil treatment significantly reduced the IOP in glaucomatous eyes.

### 2) Comparison of the Neuroprotective Effect of Galantamine and Donepezil on RGCs in Experimental Glaucoma.

The effect of galantamine on the neuroprotection of retinal ganglion cells (RGC) was compared to that of donepezil.

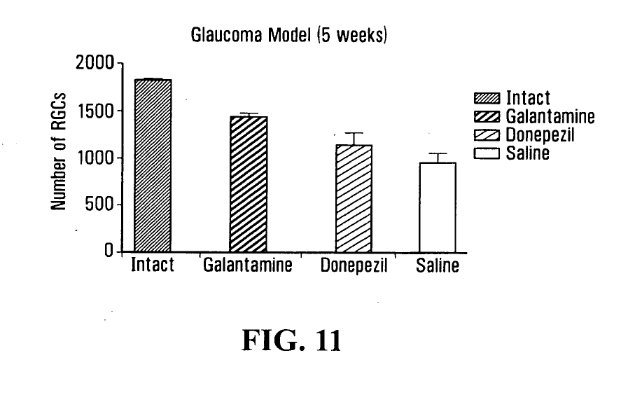

FIG. 11 shows the quantitative analysis of the number of retinal ganglion cells (RGCs, mean±S.E.M.) per mm² of retina after treatment with galantamine (3.5 mg/kg b.w., n=5), donepezil (3.5 mg/kg b.w., n=5), or vehicle (control, n=5) at 5 weeks after ocular hypertension surgery. Galantamine, donepezil or saline were administered daily by intraperitoneal injections. Galantamine treatment protected a higher number of retinal ganglion cells in experimental glaucoma than donepezil or saline.

FIG. 11 therefore shows that while both galantamine and donepezil intraperitoneal injections confer neuroprotection on retinal ganglion cells in glaucoma, galantamine surprisingly appears to be more efficacious than donepezil at the same dose, despite the IOP-reducing effect of donepezil (see Table I).

These data clearly demonstrate that as different AchE inhibitors have different physiological effects (donepezil has an IOP-reducing effect while galantamine lacks such effect), it is not obvious that all AchE inhibitors would have a neuroprotective effect on RGCs.

### 3) Comparison of the Neuroprotective Effect of Galantamine and Donepezil on RGCs After Traumatic Nerve Injury (Axotomy)

The neuroprotective effect of galantamine or donepezil was compared in a model of traumatic optic nerve injury that results in complete transection of retinal ganglion cell axons (axotomy). The advantage of this model is that retinal ganglion cell death is a consequence of axonal transection and not high intraocular pressure, thereby allowing evaluation of neuroprotection in a pressure-independent manner.

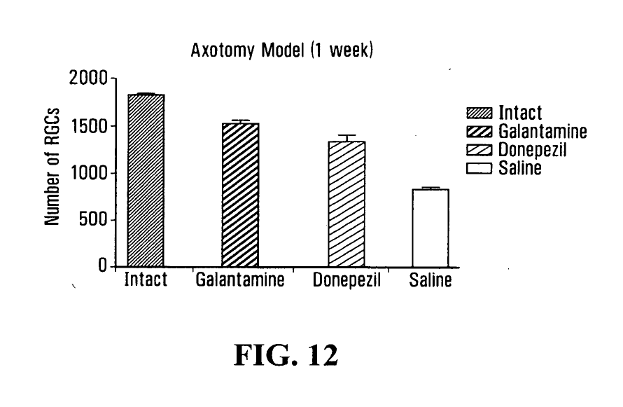

FIG. 12 shows the quantitative analysis of the number of retinal ganglion cells (RGCs, mean±S.E.M.) per mm² of retina after treatment with galantamine (3.5 mg/kg b.w., n=5), donepezil (3.5 mg/kg b.w., n=5), or vehicle (control, n=5) at 1 week after optic nerve axotomy. Galantamine, donepezil or saline were administered by independent, single intraocular injection. Galantamine treatment protected a higher number of retinal ganglion cells in the axotomy model than donepezil or saline.

These data clearly indicate that while both galantamine (3.5 mg/kg b.w.) and donepezil (3.5 mg/kg b.w.) are neuroprotective after axotomy, galantamine protects a larger number of neurons from injury-induced death than donepezil or saline.

### REFERENCES

Bores G M, Huger F P, Petko W, Mutlib A E, Camacho F, Rush D K, Selk D E, Wolf V, Kosley R. W. J, Davis L, Vargas H M. 1996. Pharmacological evaluation of novel Alzheimer's disease therapeutics: Acetylcholinesterase inhibitors related to galanthamine. Journal of Pharmacology and Experimental Therapeutics 277:728-738.
Cheng L, Sapieha P, Kittlerová P, Hauswirth W W, Di Polo A. 2002. TrkB gene transfer protects retinal ganglion cells from axotomy-induced death in vivo. J Neurosci 22:3977-3986.
Geerts H, Guillaumat P-O, Grantham C, Bode W, Anciaux K, Sachak S. 2005. Brain levels and acetylcholinesterase inhibition with galantamine and donepezil in rats, mice, and rabbits. Brain Research 1033:186-193.
Honig M G, Hume R I. 1986. Fluorescent carbocyanine dyes allow living neurons of identified origin to be studied in long-term cultures. J Cell Biol 103:171-187.
Linden R M, Perry V H. 1983. Massive retinotectal projection in rats. Brain Res 272:145-149.
Moore C G, Johnson E C, Morrison J C. 1996. Circadian rhythm of intraocular pressure in the rat. Curr Eye Res 15:185-191.
Morrison J C, Moore C G, Deppmeier L M H, Gold B G, Meshul C K, Johnson E C. 1997. A rat model of chronic pressure-induced optic nerve damage. Exp Eye Res 64:85-96.
Quigley H A. 1999. Neuronal death in glaucoma. Prog Retin Eye Res 18:39-57.
Samochocki M, Hoffle A, Fehrenbacher A, Jostock R, Ludwig J, Christner C, Radina M, Zerlin M, Ullmer C, Pereira et al. 2003. Galantamine is an allosterically potentiating ligand of neuronal nicotinic but not of muscarinic acetylcholine receptors. The Journal Of Pharmacology And Experimental Therapeutics 305:1024-1036.
Sapieha P S, Peltier M, Rendahl K G, Manning W C, Di Polo A. 2003. Fibroblast growth factor-2 gene delivery stimulates axon growth by adult retinal ganglion cells after acute optic nerve injury. Mol Cell Neurosci 24:656-672.
Schwartz M, Yoles E, Levin L A. 1999. ‘Axogenic’ and ‘somagenic’ neurodegenerative diseases: definitions and therapeutic implications. Mol Med Today 5:470-473.
Thomsen T, Kewitz H. 1990. Selective inhibition of human acetylcholinesterase by galanthamine in vitro and in vivo. Life Sciences 46:1553-1558.
U.S. Pat. No. 6,638,925, Czollner, et al., Benzazepine derivatives, medicaments containing the same and their use to prepare medicaments.
Vidal-Sanz M, Villegas-Perez M P, Bray G M, Aguayo A J. 1988. Persistent retrograde labeling of adult rat retinal ganglion cells with the carbocyanine dye diI. Exp Neurol 102:92-101.

1. A method of neuroprotecting retinal ganglion cells comprising administering to a subject in need thereof a therapeutically or prophylactically effective amount of galantamine, or a derivative, stereoisomer, analog or pharmaceutically acceptable salt thereof.

2. The method according to claim 1, wherein the galantamine or derivative, stereoisomer, analog or pharmaceutically acceptable salt thereof has the formula

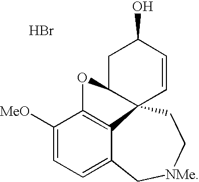

3. The method according to claim 1, wherein the galantamine or derivative, stereoisomer, analog or pharmaceutically acceptable salt thereof comprises (−)-galantamine.

4. The method according to claim 1, wherein the galantamine or derivative, stereoisomer, analog or pharmaceutically acceptable salt thereof is derived from a natural source.

5. The method according to claim 1, wherein the administering treats brain or spinal cord injury.

6. The method according to claim 1, wherein the administering treats or prevents glaucoma.

7. The method according to claim 1, wherein the administering treats or prevents age related macular degeneration.

8. The method according to claim 1, wherein said galantamine or derivative, stereoisomer, analog or pharmaceutically acceptable salt thereof is co-administered to said subject with another therapeutic agent or adjuvant therapy used to treat glaucoma or age related macular degeneration.

9. The method according to claim 1, wherein said galantamine or derivative, stereoisomer, analog or pharmaceutically acceptable salt thereof is administered by a transepidermal patch to said subject.

10. The method according to claim 1, wherein said galantamine or derivative, stereoisomer, analog or pharmaceutically acceptable salt thereof is administered orally to said subject.

11. The method according to claim 1, wherein said galantamine or derivative, stereoisomer, analog or pharmaceutically acceptable salt thereof is administered systemically to said subject.

12. The method according to claim 1, wherein said galantamine or derivative, stereoisomer, analog or pharmaceutically acceptable salt thereof is administered intraocularly or using an intraocular device to said subject.

13. The method according to claim 1, wherein said galantamine or derivative, stereoisomer, analog or pharmaceutically acceptable salt thereof is administered periocularly to said subject.

14. The method of claim 1, wherein the galantamine is administered at a daily dosage of about 0.5 mg/kg body weight to about 10 mg/kg body weight.

15-20. (canceled)
